(19) United States
(12) Patent Application Publication
(10) Pub. No.: US 2010/0016932 A1
(43) Pub. Date: Jan. 21, 2010

(54) APPARATUS AND METHOD FOR INTERNAL HYPOTHERMIC RADIOPROTECTION

(76) Inventor: Irving WEINBERG, Besthesda, MD (US)

Correspondence Address: BARNES & THORNBURG LLP, 750-17TH STREET NW, SUITE 900, WASHINGTON, DC, 20006-4675, US

(21) Appl. No.: 12/502,686

(22) Filed: Jul. 14, 2009

Related U.S. Application Data
(60) Provisional application No. 61/080,887, filed on Jul. 15, 2008.
(60) Provisional application No. 61/115,809, filed on Nov. 18, 2008.

(51) Int. Cl. A61F 7/12; A61M 36/12

(52) U.S. Cl. 607/105; 600/7

(57) ABSTRACT

A radioprotection device includes a body positionable in a living organism adjacent to both healthy and unhealthy tissue, where the living organism has a predetermined body temperature. The device includes a cooling element disposed within the body. The cooling element permits cooling of the healthy tissue adjacent to the body to a protective temperature greater than 0° C. and less than the predetermined body temperature during a therapeutic time period during which radiation is applied to the unhealthy tissue. The body discourages cooling of the unhealthy tissue to the protective temperature during the therapeutic time period, thereby providing greater radioprotection to the healthy tissue than the unhealthy tissue during the therapeutic time period.

### CROSS-REFERENCE TO RELATED APPLICATION(S)

This is a U.S. Non-Provisional patent application that relies for priority on U.S. Provisional Patent Application Ser. No. 61/080,887, filed on Jul. 15, 2008, and U.S. Provisional Patent Application Ser. No. 61/115,809, filed on Nov. 18, 2008, the contents of both of which are incorporated herein by reference.

### FIELD OF THE INVENTION

The present invention concerns an apparatus and a method for protecting normal and/or critical tissues near to a targeted cancer from radiation damage by reducing the effects of radiation on the normal and/or critical tissues. Specifically, the present invention cools tissue in a localized fashion, which reduces the deleterious effects of the radiation on the cooled normal and/or critical tissues.

### DESCRIPTION OF RELATED ART

The term “radiation” encompasses emissions associated with several nuclear processes when radioactive materials decay. Radioactive emissions encompass both photonic emissions and particle emissions.

When referring to photonic emissions, the term “radiation” typically encompasses high-energy electromagnetic photons such as x-rays and gamma rays, among others.

When referring to particle emissions, the term “radiation” encompasses decay processes such as alpha and beta decay. Alpha decay refers to the emission of energetic alpha particles from a radioactive material. The alpha particles are the nuclei of helium atoms that have been stripped of their associated electrons. Beta decay refers to the emission of energetic electrons (or positrons) from a radioactive material.

Despite obvious harmful effects of radiation, there are beneficial effects as well, especially when radiation is used in the context of medicine, either for tissue analysis or tissue treatment.

A common application of radiation for analytical purposes includes the use of simple x-rays. When x-rays are applied to tissue, the internal structure of the tissue may be revealed in a non-invasive manner, permitting a practitioner to diagnose a particular ailment, for example.

A common application of radiation for treatment includes the application of radiation to treat maladies, such as cancers, by destroying unhealthy tissues. In this example, the radiation typically is concentrated on undesirable (unhealthy) tissues to kill the unhealthy tissues while preserving the viability of desirable (healthy) tissues. As should be apparent, higher doses of radiation are applied to the unhealthy tissue than to the healthy tissue.

With respect to radiation therapies, not only may alpha, beta, gamma, and x-rays be applied, it is also known to use proton therapy.

Proton therapy is a type of radiation therapy where an energetic beam of protons are accelerated toward a target, such as a cancerous tumor. Like other forms of radiation therapy, protons damage the DNA in cells, causing cellular death. Cancerous cells, at least in part due to their high rate of division and their reduced ability for repair, are particularly vulnerable to attack on their DNA by proton beams.

When high-energy photons are applied, multiple low dose beams may be focused from several directions so that the cumulative dose at the focal point is lethal to the tissue at that location.

When particulate radiation is applied, an alpha-source, a beta source, or a proton beam source may be placed near the unhealthy tissue so that the radioactive particles impinge primarily upon the unhealthy tissue.

As should be appreciated by those skilled in the art, there is always concern that healthy tissue may be destroyed along with the unhealthy tissue.

A basic tenet of radiation therapy is to maximize the lethality of dose to target tissues, while minimizing damage in critical issues and organs.

Proton therapy has been promoted because of its ability to reduce the dose to tissues that are beyond targeted cancers.

While various techniques have been developed to minimize damage to healthy tissue when applying radiation as a therapy, continued interest exists in further ways to better protect healthy tissue.

### SUMMARY OF THE INVENTION

The present invention provides an apparatus and a method for protecting tissue from the damaging effects of radiation, thereby helping to preserve healthy tissue.

The present invention provides a radioprotection device that includes a body positionable in a living organism adjacent to both healthy and unhealthy tissue, where the living organism has a predetermined body temperature. The device also includes a cooling element disposed within the body. The cooling element permits cooling of the healthy tissue adjacent to the body to a protective temperature greater than 0° C. and less than the predetermined body temperature during a therapeutic time period during which radiation is applied to the unhealthy tissue. The body discourages cooling of the unhealthy tissue to the protective temperature during the therapeutic time period, thereby providing greater radioprotection to the healthy tissue than the unhealthy tissue during the therapeutic time period.

In one contemplated embodiment, the radioprotection device provides a protective temperature of between about 10-20° C.

In another contemplated embodiment, the radioprotection device provides a protective temperature of about 15° C.

Still further, the present invention provides a radioprotective device where the protective temperature is maintained with a range of ±2° C. during the therapeutic time period.

In one contemplated embodiment, the cooling element includes a fluid path permitting coolant to flow therethrough.

In another contemplated embodiment, the radioprotection device also includes a coolant source, a coolant line connected between the coolant source and the cooling element, a waste container, and a coolant outlet line connected between the cooling element and the waste container. Coolant at the protective temperature is circulated from the coolant source to the waste container.

It is contemplated that the radioprotection device may alternatively include electrical connections to the cooling element to provide current thereto. The cooling element may be reduced to protective temperature via the Peltier effect.

In addition, the body of the radioprotection device may have an insulation layer at least partially surrounding the body to insulate the body from tissue other than the healthy tissue and the unhealthy tissue.

Similarly, an insulation layer may be included to insulate the unhealthy tissue from the cooling element, thereby discouraging the unhealthy tissue from being cooled to the protective temperature.

The radioprotection device also may incorporate a temperature probe disposed within the body to monitor temperature within both the healthy and unhealthy tissues.

The present invention also encompasses a method for protecting healthy tissue from the effects of radiation. In the method, a cooling device, containing a cooling element, is positioned in a living organism adjacent to healthy tissue and unhealthy tissue, where the living organism has a predetermined body temperature, and where the healthy tissue is to be protected from radiation damage. Via the cooling element, the healthy tissue is cooled to a protective temperature greater than 0° C. and less than the predetermined body temperature. Radiation therapy is then applied to the unhealthy tissue. The cooling element is maintained at the protective temperature during application of the radiation to the unhealthy tissue. Application of the radiation therapy is then discontinued to the unhealthy tissue. Cooling of the cooling element is discontinued after discontinuing application of the radiation therapy.

As a part of the method, it is contemplated that the protective temperature is between about 10-20° C. Preferably, the protective temperature is about 15° C. In addition, the protective temperature is maintained within a range of ±2° C. during the therapeutic time period.

For the method, the cooling element may be cooled via a coolant. Alternatively, the cooling element may be cooled via the Peltier effect.

In another contemplated embodiment, the method includes supplying the coolant to the cooling element from a coolant source, and discharging the coolant from the cooling element into a waste container.

In still another contemplated embodiment, the method includes providing insulation between at least a portion of the cooling element and the unhealthy tissue to discourage unhealthy tissue from being cooled to the protective temperature.

Additionally, the method may include monitoring the temperature within both the healthy and unhealthy tissues via a temperature probe disposed within the cooling device.

Other aspects of the present invention will become apparent from the discussion provided herein.

### BRIEF DESCRIPTION OF THE DRAWINGS

The present invention will now be described in connection with the drawings appended hereto, in which:

FIG. 1 is a schematic illustration of a first embodiment of an apparatus for providing radioprotection to tissue via cooling of the tissue according to the present invention;

FIG. 2 is a schematic illustration of a second embodiment of an apparatus for providing radioprotection to tissue via cooling of the tissue according to the present invention;

FIG. 3 is a schematic illustration of a third embodiment of an apparatus for providing radioprotection to tissue via cooling of the tissue according to the present invention;

FIG. 4 is a schematic illustration of a fourth embodiment of an apparatus for providing radioprotection to tissue via cooling of the tissue according to the present invention;

FIG. 5 is a flow diagram illustrating a first embodiment of a method according to the present invention; and

FIG. 6 is a flow diagram illustrating a second embodiment of a method according to the present invention.

### DESCRIPTION OF EMBODIMENT(S) OF THE INVENTION

While the present invention is described in connection with one or more embodiments, the present invention is not intended to be limited solely to the embodiments described. To the contrary, there are numerous equivalents and variations that should be apparent to those skilled in the art upon reading and understanding the instant disclosure. The invention is intended to encompass those equivalents and variations.

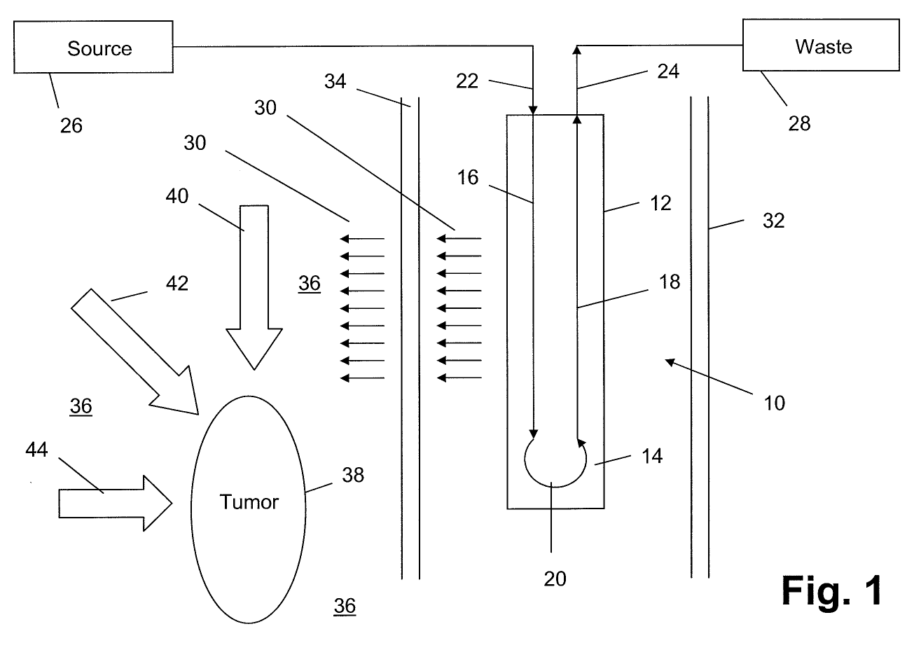

FIG. 1 is a schematic illustration of a first embodiment of the apparatus of the present invention. In its basic, simple form, the apparatus 10 of the present invention takes a complementary approach, utilizing means and methods to protect critical structure(s) in tissue from damage due to radiation delivered in the area. The specific method and apparatus cools normal (i.e., healthy) tissues during or near the time when radiation therapy is applied.

It is known that the act of cooling tissues, whether to the freezing point, or to temperatures above the freezing point, has a radio-protective effect. In the case of frozen tissues, the beneficial cooling effect has been ascribed to trapping of radicals, leading to reduced interaction with DNA. At temperatures above freezing (e.g., 15 degrees Celsius), the radioprotective effect has been ascribed to various causes, including altered DNA configurations, anti-oxidant activity, reduced metabolic rate effects, and local anoxia.

Although early reports demonstrated no beneficial effects of hypothermia in isolated mammalian cells (e.g., cell cultures), as compared to significant beneficial events in complex systems, more recent articles have shown beneficial effects even for cultured cells. Local hypothermia has been applied to the skin, resulting in reduced parasternal skin reactions after radiotherapy, for example. Local hypothermia for skin malignancies (when combined with radiotherapy) is compensated as therapy by third-party payers in certain localities.

Prior work has promoted the use of hypothermia applied externally, or to the whole-body, as a radio-protective strategy. Local hypothermic application via a heat pipe has been contemplated for treatment of inflammatory conditions, but not of cancer. Cooling tumors as a form of therapy (cryosurgery) has been shown to be relatively ineffective in ablating tumors, as compared to radiotherapy. The current invention differs substantially from cryosurgery, in that the current invention applies cold in order to protect normal tissue, and does not have a principal purpose of ablating cancer (as in cryosurgery).

It is known that under certain conditions, cooling can increase destruction of cancer cells, due to the increased capability of the cancer tissues to absorb oxygen at lower temperatures.

The invention is expected to be particularly useful in treatment of prostate cancer, where injury to the urethra, rectum, and urinary bladder is common during radiotherapy (e.g., HDR brachytherapy, external beam radiotherapy, etc.). The invention contemplates providing cooling to some or all of these organs via cannulation, at or around the time of application of radiotherapy. Another exemplary application is for patients receiving pelvic irradiation, in which the ureters could be accessed endoscopically and flushed with coolant.

In the current invention, the cooling can be delivered through a catheter (e.g., a Foley-type catheter, which can cool the urethra and/or bladder), or via irrigation into a cavity or compartment (e.g., via a rectal cannula, which can cool colonic mucosal tissues, or a spinal needle, which can cool the cerebrospinal fluids). The cooling effect also may be implemented via a needle or other heat-conducting structure that is physically connected to a cold object, or electrically via the Peltier effect.

With this basic overview in mind, the present invention will now be described in connection with one or more embodiments.

As noted above, FIG. 1 provides a schematic illustration of a first contemplated embodiment of the cooling device 10 of the present invention. The cooling device 10 includes a body 12 with a tip portion 14. To provide cooling, the body 12 includes an inlet line 16 and an outlet line 18. The inlet line 16 and the outlet line 18 are connected via a cooling loop 20 embedded within the tip portion 14 of the body 12. The inlet line 16 is connected to a cooling inlet 22, exterior to the body 12. The outlet line 18 is connected, in turn, to a cooling outlet 24, also exterior to the body 12.

In this embodiment of the present invention, it is contemplated that a coolant, such as water or saline, will be circulated from a coolant source 26 into the body 12 via the coolant inlet 22. After circulating through the coolant loop 20, the coolant is discharged into a waste container 28 via the cooling outlet 24. The temperature of the coolant will be maintained by a suitable processor or equivalent, as should be appreciated by those skilled in the art. It is also contemplated that the coolant may be circulated from the waste container 28 back to the source 26, if appropriate.

In an alternative embodiment, it is contemplated that the body 12 may be provided with a plurality of coolant inlets 22 and coolant outlets 24 to provide enhanced cooling to the body 12.

In still another alternative embodiment, it is contemplated that the inlet line 16 and the outlet line 18 within the body 12 will be arranged to maximize the cooling effect provided by the body 12. For example, the inlet line 16 and the outlet line 18 may be spirally intertwined within the body 12. For directional effects, the lines 16, 18 may be thermally isolated from one another. Still further variations are considered to be encompassed by the present invention, as should be appreciated by those skilled in the art.

As noted above, the cooling device 10 may provide electrically-based cooling via the Peltier effect. In this embodiment, the inlet line 16 and the outlet line 18 are replaced with electrical connections. The cooling loop 20 is replaced by a suitable Peltier element that provides cooling in response to the application of electrical energy thereto.

Regardless of whether the cooling device 10 relies on coolant or electricity to apply cooling to tissue from the tip portion 14, the cooling area of the tip portion 14 may be engineered for various different applications. In the embodiment illustrated in FIG. 1, the tip portion 14 generates a temperature gradient 30 along a side portion of the cooling tip 14. As should be apparent, the temperature gradient 30 may be applied along any portion of the cooling tip 14, including the region adjacent to the cooling loop 20.

With this in mind, it is contemplated that the tip portion 14 may be engineered to provide cooling to tissue in selectable regions of the body 12. This may be accomplished via control over the temperature of the coolant, control over the flow of coolant through the body 12, and/or control over the location of the Peltier effect within the body. In other words, the location and direction of the temperature gradient 30 may be carefully controlled to maximize the application of cooling to healthy tissue while minimizing the application of cooling to the unhealthy tissue to which the radiation is applied.

With further reference to FIG. 1, the body 12 is shown having been inserted into a body area, such as a colon. The side walls 32, 34 of the colon are illustrated to provide context for the discussion of the present invention. As should be apparent, the body 12 of the cooling device 10 need not be introduced into a body cavity. Instead, the body 12 may be inserted directly into an incision in the tissue itself. Alternatively, the body area may be a urethra, thecal sac, or ureter, among others.

As shown in FIG. 1, the temperature gradient 30 (or the cooled area) passes through the side wall 34 where it cools the healthy tissue 36 surrounding the unhealthy tissue 38 that is to be subjected to one or more types of radiation 40, 42, 44. The unhealthy tissue 38 may be a cancerous tumor or the like, as indicated above.

It is contemplated, in one alternative embodiment, that the coolant may be circulated outside of the body 12, in direct contact with the healthy tissue 36, if appropriate. A combination of coolant circulation within the body 12 and exterior to the body 12 also may be employed.

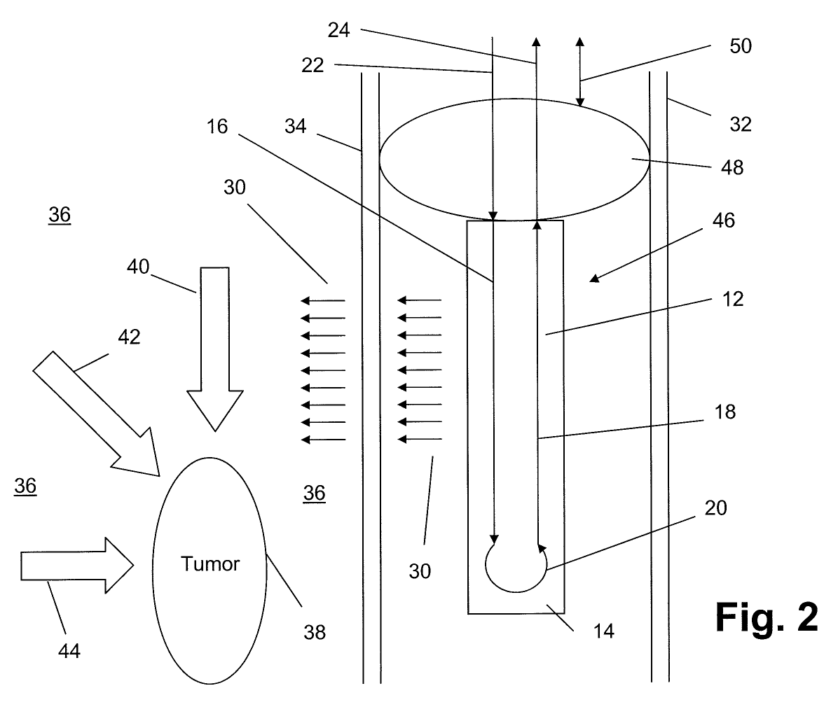

FIG. 2 is a schematic illustration of a second embodiment of the cooling device 46 according to the present invention. This embodiment is the same as the cooling device 10 illustrated in FIG. 1, with one difference.

In FIG. 2, the cooling device 46 includes a balloon 48 that helps to position the cooling device 46 by applying pressure to the side walls 32, 34 of the surrounding tissue. The balloon 48 may be desirable, especially in instances where the cooling device 46 has been inserted into a body cavity through which a fluid flows. The balloon 48 is expected to secure the cooling device 46 so that cooling is applied to the healthy tissue 36. So that the balloon 48 may be inflated and deflated, an access line 50 is provided.

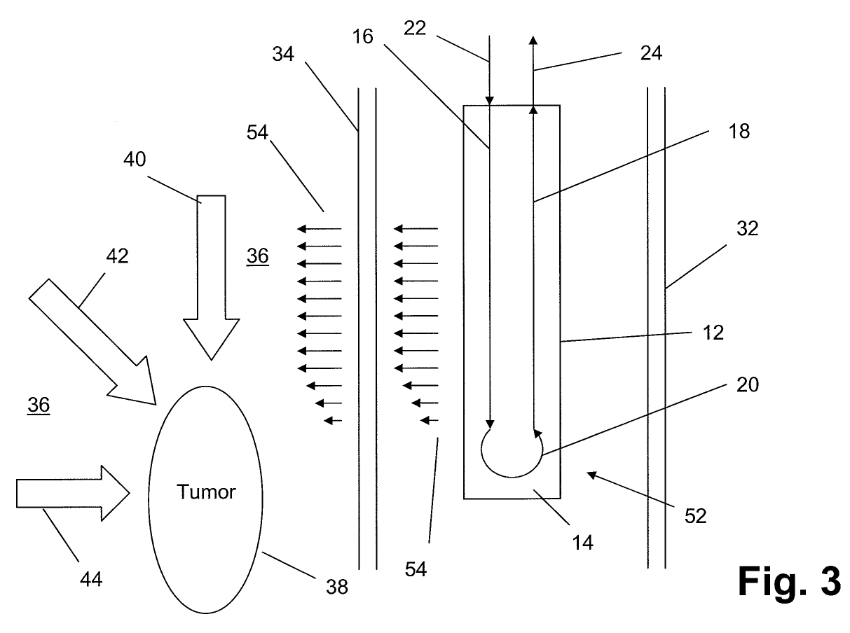

FIG. 3 is a schematic illustration of a third embodiment of the cooling device 52 of the present invention. In this illustration, the cooling device is the same as described in FIG. 1, except that the temperature gradient 54 differs from the temperature gradient 30 associated with the cooling device 10.

With respect to the cooling device 52, the temperature gradient 54 is a variable temperature gradient. As a result, with this embodiment, it is contemplated that a greater cooling may be applied to tissue at a predetermined distance from the unhealthy tissue 38 than the healthy tissue 36 immediately adjacent to the unhealthy tissue 38. As a result, due to the greater amount of cooling, the healthy tissue 36 further from the unhealthy tissue 38 will be afforded a greater radioprotection than the healthy tissue 36 immediately adjacent to the unhealthy tissue 38.

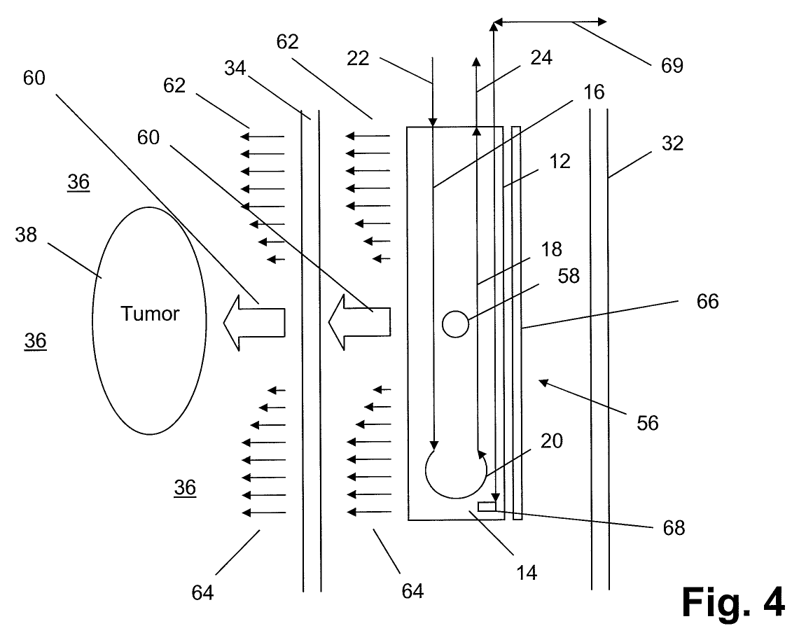

FIG. 4 provides a schematic illustration of a fourth embodiment of the cooling device 56 of the present invention. This embodiment is similar to the embodiment illustrated in FIG. 3, with a few notable differences. The cooling device 56 incorporates a source of radiation 58 to direct radiation 60 to the unhealthy tissue 38. The cooling device also generates two separate cooling zones, each with a variable temperature gradient 62, 64. In this embodiment, the device 56 is responsible both for cooling radioprotection and for the radiation 60.

As with the embodiment of the cooling device 52 illustrated in FIG. 4, the temperature gradients 62, 64 are variable gradients that afford a greater amount of radioprotection for healthy tissue 36 disposed a greater distance from the unhealthy tissue 38.

FIG. 4 also illustrates a thermal barrier 66 or insulator adjacent to the body 12 of the cooling device 56. This barrier may be added to insulate portions of the device 56 from areas not requiring cooling.

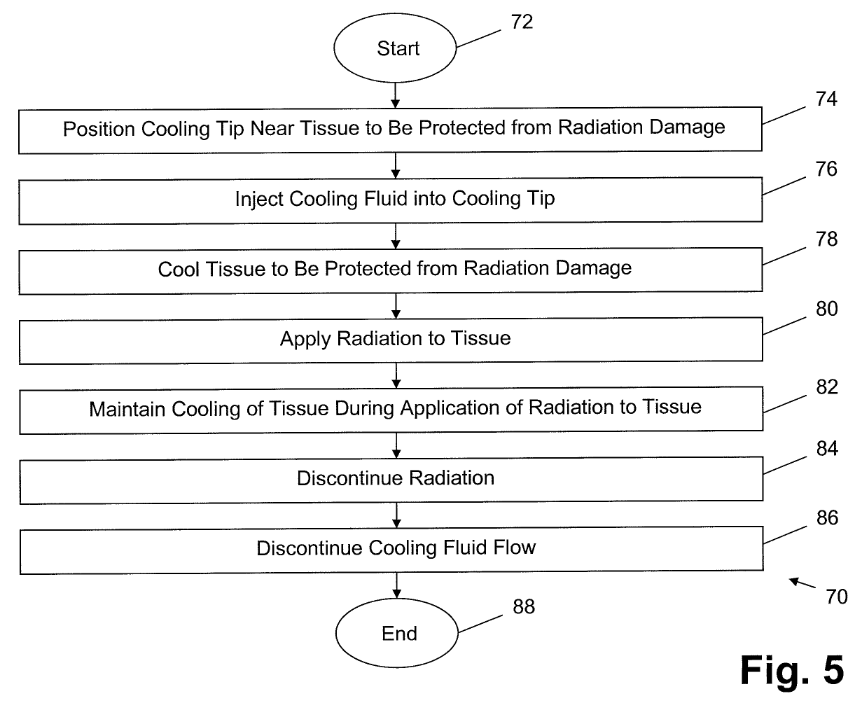

FIG. 5 illustrates a first radioprotection method 70 contemplated by the present invention.

As illustrated in FIG. 5, the radioprotection method 70 begins at 72. At 74, the method 70 proceeds to the step of positioning the tip portion 14 near healthy tissue 36 to be protected from radiation damage. Then, the method 70 proceeds to step 76, where coolant is injected into the tip portion 14. At step 78, the tissue 36 is cooled such that it is protected from damage by radiation 40, 42, 44, 60. The healthy tissue 36 is cooled to a temperature where the tissue 36 is protected from radiation damage. The temperature may differ depending upon the tissue type, density, etc. At step 80, radiation 40, 42, 44, 60 is applied to the unhealthy tissue 38. At step 82, cooling of the healthy tissue 36 is maintained during application of the radiation 40, 42, 44, 60 to the unhealthy tissue 38. At step 84, the radiation 40, 42, 44, 60 is discontinued. Afterwards, at step 86, cooling fluid flow is discontinued. The method 70 ends at 88.

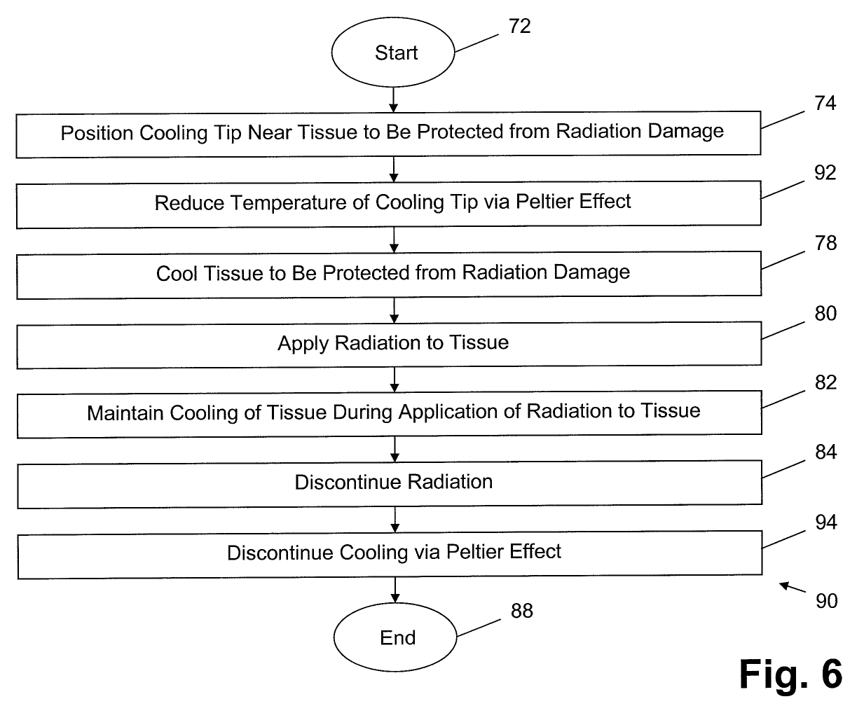

FIG. 6 provides a second radioprotection method 90 contemplated by the present invention. This method 90 is the same as the method 70, with two specific changes. First, at step 92, the temperature of the cooling tip 14 is reduced using the Peltier effect. Second, at step 94, the cooling via the Peltier effect is discontinued. In all other respects the method 90 is the same as the method 70.

It is contemplated that a cannula, catheter, stent, or other similar medical device may be used for the body 12 of the cooling device 10, 46, 52, 56. Devices that may be used include, but are not limited to 4-french coronary artery stents, 4.5 pigtail catheters, Foley-type catheter, and/or polyethylene PE-50 catheters. As should be appreciated by those skilled in the art, a wide variety of existing devices may be adapted to incorporate the cooling device 10, 46, 52, 56 of the present invention.

It is contemplated, in one embodiment, that the coolant may be a saline solution. Other fluids also may be used without departing from the scope of the present invention.

With respect to the temperature to which the healthy tissue is cooled for radioprotection, it is contemplated that the tissue may be cooled to a temperature below body temperature but above freezing. It is expected that the tissue will be maintained above freezing to avoid causing cellular damage. The exact temperature employed, however, is expected to differ depending upon the location of the unhealthy tissue 38 and the type of healthy tissue 36 to be protected.

As should be appreciated by those skilled in the art, the freezing point of water is 0° C. and the temperature of the human body is 37° C. Accordingly, in its most broad application, the cooling device 10, 46, 52, 56 of the present invention will cool the healthy tissue 36 to a temperature between about 0-37° C. Concerning the lower limit of freezing, it is understood that tissue may have a freezing temperature below 0° C. However, since the present invention does not contemplate cooling healthy tissue 36 to the freezing point, 0° C. is expected to be the lower limit for cooling of the healthy tissue.

The cooling device 10, 46, 52, 56 of the present invention preferably cools the healthy tissue to a temperature of between about 10-20° C., with a temperature of about 15° C., being the most preferred. A temperature of about 15° C. is expected to provide sufficient radioprotection without endangering the viability of the tissue over an extended period of time.

Radiation therapies may be applied to unhealthy tissue for periods of time encompassing several minutes and/or several hours, depending upon the total dose required for the particular therapy. The cooling device 10, 46, 52, 56 of the present invention, therefore, will maintain the body 12 at a temperature of 10-20° C. for the entire duration of the radiation therapy. For maximum effect, the cooling device 10, 46, 52, 56 will not deviate more that 2° C. from the required temperature. As a result, where the selected temperature is about 15° C., the cooling device 10, 46, 52, 56 will maintain the temperature of the healthy tissue in a range of 13-17° C.

To assure that the cooling device 10, 46, 52, 56 is maintained at a constant temperature or within a range of temperatures, the body 12 may be provided within one or more temperature sensors that are connected to a processor. Upon receipt of the temperature information from the temperature sensors, the processor will adjust the flow of coolant or the current applied to maintain the temperature of the cooling device 10, 46, 52, 46 within the specified range.

It is also contemplated that the healthy tissue 36 may be treated chemically to resist the effects of the cooling applied thereto. For example, the tissue 36 may be pretreated with a substance that increases the tissue's resistance to freezing.

It is also contemplated that a temperature probe or method may be employed to monitor operation of the method and apparatus, such as magnetic resonance imaging, infrared imaging, or local thermometric sensors. For example, a temperature probe 68, as illustrated in FIG. 4, may be supplied to monitor the temperature of the tissue 36, among other temperature parameters within the organism. The temperature probe 68 may be connected to a processor via a communication line 69. The temperature probe 68 may cooperate with one or more other devices, such as magnetic resonance imager, infrared imager, or the like. The temperature sensor, whether it be the temperature probe 68, a magnetic resonance imager, or a local thermometric sensors, does not need to be incorporated into the body 12 of the cooling device 10, 46, 52, 56.

As should be appreciated by those skilled in the art, the present invention is not intended to be limited to the embodiments described herein. There are numerous variations and equivalents that are intended to be encompassed thereby.

What is claimed is:

1. A radioprotection device, comprising:
a body positionable in a living organism adjacent to both healthy and unhealthy tissue, wherein the living organism has a predetermined body temperature; and
a cooling element disposed within the body;
wherein the cooling element permits cooling of the healthy tissue adjacent to the body to a protective temperature greater than 0° C. and less than the predetermined body temperature during a therapeutic time period during which radiation is applied to the unhealthy tissue, and
wherein the body discourages cooling of the unhealthy tissue to the protective temperature during the therapeutic time period, thereby providing greater radioprotection to the healthy tissue than the unhealthy tissue during the therapeutic time period.

2. The radioprotection device of claim 1, wherein the protective temperature is between about 10-20° C.

3. The radioprotection device of claim 2, wherein the protective temperature is about 15° C.

4. The radioprotection device of claim 1, wherein the protective temperature is maintained with a range of ±2° C. during the therapeutic time period.

5. The radioprotection device of claim 1, wherein the cooling element includes a fluid path permitting coolant to flow therethrough.

6. The radioprotection device of claim 5, further comprising:
a coolant source;
a coolant line connected between the coolant source and the cooling element;
a waste container; and
a coolant outlet line connected between the cooling element and the waste container,
wherein coolant at the protective temperature is circulated from the coolant source to the waste container.

7. The radioprotection device of claim 1, further comprising:
electrical connections to the cooling element to provide current thereto,
wherein the cooling element is reduced to protective temperature via the Peltier effect.

8. The radioprotection device of claim 1, wherein the body further comprises:
an insulation layer at least partially surrounding the body to insulate the body from tissue other than the healthy tissue and the unhealthy tissue.

9. The radioprotection device of claim 1, wherein the body further comprises:
an insulation layer to insulate the unhealthy tissue from the cooling element, thereby discouraging the unhealthy tissue from being cooled to the protective temperature.

10. The radioprotection device of claim 1, further comprising:
a sensor to monitor temperature within both the healthy and unhealthy tissues.

11. A method for protecting healthy tissue from the effects of radiation, comprising:
positioning a cooling device, containing a cooling element, in a living organism adjacent to healthy tissue and unhealthy tissue, wherein the living organism has a predetermined body temperature, and wherein the healthy tissue is to be protected from radiation damage;
cooling, via the cooling element, the healthy tissue to a protective temperature greater than 0° C. and less than the predetermined body temperature;
apply radiation therapy to the unhealthy tissue;
maintaining the cooling element at the protective temperature during application of the radiation to the unhealthy tissue;
discontinuing application of the radiation therapy to the unhealthy tissue; and
discontinuing cooling of the cooling element after discontinuing application of the radiation therapy.

12. The method of claim 11, wherein the protective temperature is between about 10-20° C.

13. The method of claim 12, wherein the protective temperature is about 15° C.

14. The method of claim 11, wherein the protective temperature is maintained within a range of ±2° C. during the therapeutic time period.

15. The method of claim 11, wherein the cooling element is cooled via a coolant.

16. The method of claim 11, wherein the cooling element is cooled via the Peltier effect.

17. The method of claim 15, further comprising:
supplying the coolant to the cooling element from a coolant source; and
discharging the coolant from the cooling element into a waste container.

18. The method of claim 11, further comprising:
providing insulation between at least a portion of the cooling element and the unhealthy tissue to discourage unhealthy tissue from being cooled to the protective temperature.

19. The method of claim 11, further comprising:
monitoring temperature within both the healthy and unhealthy tissues.
